(19) United States
(12) Patent Application Publication
(10) Pub. No.: US 2007/0253980 A1
(43) Pub. Date: Nov. 1, 2007

(54) New pharmaceutical composition from natural materials for regulating immunity, its preparation method and use

(76) Inventor: Jorge P. Chang Cabezas, Beijing (CN)

Correspondence Address: ANDREWS KURTH LLP;Intellectual Property Department, Suite 1100, 1350 I Street, N.W., Washington, DC, 20005, US

(21) Appl. No.: 11/819,479

(22) Filed: Jun. 27, 2007

Related U.S. Application Data
(62) Division of application No. 11/451,458, filed on Jun. 13, 2006.

(30) Foreign Application Priority Data
Apr. 21, 2006 (CN) 200610072873.9

(51) Int. Cl. A61K 39/21; A61K 125/00; A61K 127/00; A61K 135/00; A61K 36/06; A61P 37/00; A61P 43/00; A61P 31/18; A61K 36/481; A61K 36/605; A61K 39/00

(52) U.S. Cl. 424/208.1; 424/184.1; 424/195.15; 424/757; 424/773; 424/774; 424/779

(57) ABSTRACT

The present invention relates to a new pharmaceutical composition from natural materials belonging in traditional Chinese medicine and to its preparation method. Said new pharmaceutical composition comprises Astragalus root, Pleurotus fungi, Winnged Euonymus Twig and Mulberry Leaf. At first, astragalus root, Pleurotus fungi, Winnged Euonymus Twig and Mulberry Leaf are extracted by water or alcohol to obtain extractive. The obtained extractive can be got by extracting the above four materials together, or by combining the extractive from the three materials together (Astragalus root, Winnged Euonymus Twig and Mulberry Leaf) among above four materials with the extractive from Pleurotus fungi singly. Then the mixed extractive is centrifugalized and separated. If need be, the gained centrifugate can be made into oral formulations in various dose form with excipient by routine method. Said pharmaceutical composition has the effect of regulating immunity on the body. It has been tested and found that said pharmaceutical composition has no toxicity and has good curative effect on AIDS, diabetes and cancer.

### TECHNICAL FIELD

The present invention relates to traditional Chinese medicine field, and especially to a new pharmaceutical composition from natural materials, which belong in traditional Chinese medicine, for regulating immunity.

### BACKGROUND OF THE INVENTION

The term “immunity” means the function of the body on the basis of immune components, such as immune organs and immunoglobulins, to search and recognize the antigen. That is, it means the function of the body to distinguish self and non-self, thereby defending against diseases and maintaining the physiological balance of the body. It can recognize normal and abnormal microorganisms and cells through human immune system and exclude microorganisms both in vitro and vivo that may cause disease and denatured and senescent cells through immune response. In brief, immunity means the ability of the immune system to protect our bodies, so that it can be a measure index of immune function.

T cells belong to leucocyte cells and play an important role in the immune system. There are two main T cells in the human body, one of which is called CD4 cell on the surface of T cells. The CD4 cell is also called helper@ of the immune system, which can command the body defending against microorganisms, such as viruses. According to a World Health Organization (WHO) report, the value of CD4 cells in normal human body is between 700 and 1100 cells/mm³, to a certain extent, which shows the normal value of human immunity. Generally when the value of CD4 is out of said range, the immune function of human body is either over-reactive or low, viz. immune dysfunction, which may cause diseases.

The function of the immune system is to search, recognize and attack substances and pathogens such as bacteria and viruses that may cause disease. Various diseases may be caused when the function of the immune system is disordered, that is, the human immunity is abnormnal. For example, an over-reactive immune reaction may cause autoimmune diseases such as diabetes, and immunodeficiency diseases may be caused when the immunofunction is low or deficient. When the function of the immune supervision is disordered, the value of immunity is lower than the normal value, the immune system could not recognize and clear mutative cells inside the body in time. That is the main cause and determinant of tumor and cancer. That is, diseases may be caused when the immune function of human body is either too high or too low so that regulating immunity is very important to ensure human health.

The whole name of AIDS is Acquired Immune Deficiency Syndrome@. AIDS belongs to infectious diseases caused by AIDS: virus, human immunodeficiency virus (HIV), which intrudes into the human body, destroys the immunofunction and then causes a series of cureless infection and tumor and results in the death finally. Objects attacked by AIDS virus are CD4 cells and then AIDS virus will achieve the destruction to immune system of human body through the attack. The immune system of human body may be suppressed and the immunofunction may be destroyed when the value of CD4 cells is decreased to a certain extent and the value of CD8 cells maintains still high level, so that the measure value of CD4 has an important effect on judging the curative effect of AIDS and the efficacy of immune system of the patients. “Viral Load”, the amount of HIV, means the “number of copies” of HIV RNA in the blood, which shows the propagation level of viruses and is an important index to detect infection, to direct therapy and to show prognosis and curative effect. Recently, to achieve the aim of curing AIDS, the treatment tactics for AIDS all over the world is decreasing Viral Load by all means and reestablishing and/or maintaining immune function (the increase of CD4 cells count), and improving the life quality and prolonging the life of AIDS patients.

The drugs treating AIDS at prior arts take an effect by killing HIV. At the sometime, these drugs will also kill CD4 cells and then will decrease the amount of the CD4, instead of increasing the amount of CD4 cells. Therefore the drugs could not improve the immune function of patients and individuals with the deadly virus.

The symptoms of diabetes include mainly high blood sugar and also include excessive thirst, excessive urination and weight loss and so on. Diabetes has several complications like fatigue, infection, paresthesia (such as numbness, titillation, formication or pins-and-needles etc.) and blurred vision. These complications have an effect on a lot of organs throughout the body and cause even the disability and death further. They threaten the health of human being greatly.

Cancer is also called malignancy. Common cancers include cervical cancer, lung cancer, neuroblastoma, liver cancer, colon cancer, all of which are deadly diseases.

Up to now, it is not reported that, any pharmaceutical composition in traditional Chinese medicine could regulate immunity, and especially could cure diseases caused by immune dysfunction, such as AIDS, Diabetes and Cancer and so on.

### DISCLOSURE OF THE INVENTION

An object of the present invention is to provide a new pharmaceutical composition from natural materials, which belong in traditional Chinese medicine, for regulating immunity.

Another object of the present invention is to provide the preparation method of said pharmaceutical composition.

Another object of the present invention is to provide the use of said pharmaceutical composition.

Another object of the present invention is to provide a method of regulating immunity by using said pharmaceutical composition.

Another object of the present invention is to provide a treatment method of diseases caused by immune dysfunction with said pharmaceutical composition.

Another object of the present invention is to provide a treatment method of AIDS with said pharmaceutical composition.

Another object of the present invention is to provide a treatment method of diabetes with said pharmaceutical composition.

The objects of the present invention are carried out through following technical solutions:

The present invention provides a new pharmaceutical composition for regulating immunity. Said pharmaceutical composition comprises astragalus root, Pleurotus fungi, Winnged Euonymus Twig and Mulberry Leaf, specially, the composition comprises 5-60 parts by weight of Astragalus root, 10-100 parts by weight of Pleurotus fungi, 10-100 parts by weight of Winnged Euonymus Twig and 3-40 parts by weight of Mulberry Leaf, preferably, 10-30 parts by weight of Astragalus root, 20-50 parts by weight of Pleurotus fungi, 20-50 parts by weight of Winnged Euonymus Twig and 5-20 parts by weight of Mulberry Leaf, more preferably, 15-25 parts by weight of Astragalus root, 30-40 parts by weight of Pleurotus fungi, 30-40 parts by weight of Winnged Euonymus Twig and 10-15 parts by weight of Mulberry Leaf, most preferably, 20 parts by weight of Astragalus root, 35 parts by weight of Pleurotus fungi, 35 parts by weight of Winnged Euonymus Twig and 12 parts by weight of Mulberry Leaf.

Said astragalus root, Pleurotus fungi, Winnged Euonymus Twig and Mulberry Leaf can be purchased from market.

Astragalus root, Latin name Astragalus membranaceus, belongs to perennial herb plant and is also called Astragalus membranaceus Bunge var mongolicus (Bunge) Hsiao, which can be purchased from drugstore and market.

Pleurotus fungi mean a family of some fungi having similar property and active ingredients (chenshiyu, chenhaiying, et al., Collection of Mushroom Prescriptions, Shanghai Science and Technology Press, Jan. 1, 2000, page 422425), which include mainly Pleurotus ostreatus (also called Pleurotus spp.), Pleurotus sapidus, Pleurotus sajor and so on. Anyone or any their mixture of said Pleurotus fungi can be used in the present invention. Pleurotus fungi distribute everywhere in china and are mainly cultivated artificially now.

The Latin name of Winnged Euonymus Twig is Euonymus alatus.

The Latin name of Mulberry Leaf is Folium Mori.

The weight ratio of above materials, astragalus root, Pleurotus fungi, Winnged Euonymus Twig and Mulberry Leaf, is based on the raw materials in the dry state. Of course, the weight ratio of fresh raw materials can be counted according to the ratio of water loss in the dry process of fresh raw materials.

The Said pharmaceutical composition can be made to oral formulations, such as granules, tablets, capsules, powders, oral liquid and pills, preferably granules and oral liquid.

The present invention provides the preparation method, of said pharmaceutical composition. At first, astragalus root, Pleurotus fungi, Winnged Euonymus Twig and Mulberry Leaf are extracted by water or alcohol to obtain extractive. The obtained extractive can be got by extracting the above four materials together, or combining the extractive from the three materials together (stragalus root, Winnged Euonymus Twig and Mulberry Leaf) among above four materials with the extractive from Pleurotus fungi singly. Then the mixed extractive is centrifugalized and filtered. If need be, the gained centrifugate can be made into the oral formulations in various dose form with some excipient by routine method. For example, the centrifugate is concentrated and then dried by spray to powder, or the centrifugate is concentrated and then dried and crushed to fine powder and then the powder is made into granules with proper amount of excipient, or the centrifugate is concentrated and then dried by spray and pelletized to be granules.

The administration dose of said pharmaceutical composition, if converting the composition into it's the crude drug (natural materials), is at least 8 g crude drug/day/person (60 kg). Said pharmaceutical composition can regulate immune function and cure diseases caused by immune dysfunction in said dose. The drugs can have a good curative effect when the dose is in the rage of 20-100 g crude drug/day/person (60 kg). The curative effect does not improve greatly when the dose is above 100 g crude drug/day/person (60 kg). According to under-mentioned study on pharmacology and toxicology, the dose can not have a toxic effect on the patients. Said pharmaceutical composition can have an effect on health-care of patients when it is used for long term in a lower dose, such as below 8 g crude drug/day/person (60 kg).

The present invention provides a new pharmaceutical composition for regulating immunity that can cure and prevent various diseases caused by immune dysfunction, such as AIDS, diabetes and cancer and so on, and have a good curative effect. It has been tested and found that said pharmaceutical composition has no toxicity

The present invention provides a method of regulating immunity. The method includes that the patients suffered from immune dysfunction, if need be, are administrated with a pharmaceutically effective amount of the new pharmaceutical composition according to the present invention.

The present invention provides a treatment method of diseases caused by immune dysfunction. The method includes that the patients suffered from said diseases, if need be, are administrated with a pharmaceutically effective amount of the new pharmaceutical composition according to the present invention.

The present invention provides a treatment method of AIDS. The method includes that the patients suffered from said disease (or individuals with the deadly virus), if need be, are administrated with a pharmaceutically effective amount of the new pharmaceutical composition according to the present invention. The object of the treatment is to inhibit HIV virus and increase the amount of CD4 cells, that is, to maintain and improve immune function, relieve the symptoms of patients, improve the life quality of patients and prolong the life. In order to achieve above-mentioned object, preferably, the dose is no less than 20 g crude drug/day/person (60 kg).

The present invention provides a treatment method of diabetes. The method includes that the patients suffered from said disease, if need be, are administrated with a pharmaceutically effective amount of the new pharmaceutical composition according to the present invention. The object of the treatment is to decrease blood sugar, cholesterol, blood lipoids and blood pressure of patients, improve blood circulation, blood microcirculation and endocrine of patients, and relieve, improve and eliminate all kinds of symptoms and complications by regulating immune function of patients, thereby cure diabetes fundamentally. In order to achieve above-mentioned object, preferably, the dose is no less than 20 g crude drug/day/person (60 kg).

The present invention provides a treatment method of cancer. The method includes that the patients suffered from said disease, if need be, are administrated with a pharmaceutically effective amount of the new pharmaceutical composition according to the present invention. The object of the treatment is to cure cancer by regulating the immune function of cancer patients. In order to achieve above-mentioned object, preferably, the dose is no less than 20 g crude drug/day/person (60 kg).

### DESCRIPTION OF THE DRAWINGS

FIG. 1. the toxic effect of the granules of example 1 on C8166 in test II

FIG. 2. the toxic effect of AZT on C8166 in test II

FIG. 3. the inhibiting effect of the granules of example 1 on Syncytium formation of C8166 cells induced by HIV-1 in test II

FIG. 4. the inhibiting effect of AZT on Syncytium formation of C8166 cells induced by HIV-1 in test II

FIG. 5. the inhibiting effects of the granules of example 1 in different concentrations on the fusion in test II

FIG. 6. the inhibiting effects of Dextran Sulfate in different concentrations on the fusion in test II

FIG. 7. the protective effects of the granules of example 1 in different concentrations on C8166 cells induced by HIV-1 in test II

FIG. 8. the protective effects of AZT in different concentrations on C8166 cells induced by HIV-1 in test II

FIG. 9. the effects of IA and IIA on cell cycle of HeLa cells in test IV

FIG. 10. the effects of IA and IIA on cell cycle of SK cells in test IV

FIG. 11. the effects of IA and IIA on cell cycle of SY5Y cells in test IV

FIG. 12. the effects of IA and IIA on cell cycle of A549 cells in test IV

### THE BEST MODES FOR CARRYING OUT THE INVENTION

The present invention is further described by following examples, however, the present invention is not restricted to these examples.

### Example 1

### The Preparation of Granules of the Pharmaceutical Composition According to the Present Invention

200 g Astragalus root, 350 g Pleurotus ostreatus (also called Pleurotus spp.), 350 g Winnged Euonymus Twig and 120 g Mulberry Leaf were weighed respectively and decocted together for two hours with 10 L water every time and for three times. The obtained extractives were combined and filtrated. The gained filtrate was concentrated to relative density between 1.03 and 1.04 at 60□. The concentrated filtrate was stood overnight. Then the filtrate was filtrated again using a 200-mesh-filter. The obtained filtrate was centrifugalized and separated. The gained centrifugate was concentrated and then dried and crushed to powder finally. Total amount of 250 g-dextrin and/or mannitol was mixed evenly with the obtained powder and then the mixture was pelletized. 90 bags were filled with obtained granules. Each bag was filled with 5 g granules.

Direction for administration: 3 bags each time

### Example 2

### The Preparation of Powders of the Pharmaceutical Composition According to the Present Invention

150 g Astragalus root, 400 g Pleurotus ostreatus, 300 g Winnged Euonymus Twig and 150 g Mulberry Leaf were weighed respectively and decocted together for two hours with 10 L water every time and for three times. The obtained extractives were combined and filtrated. The gained filtrate was concentrated to relative density between 1.04 and 1.60 at 60□. The concentrated filtrate was stood overnight. Then the filtrate was filtrated again using a 200-mesh-filter. The obtained filtrate was centrifugalized and separated. The gained centrifugate was concentrated and then dried by spray to powder. 40 bags were filled with obtained powders. Each bag was filled with 5 g powders.

Direction for administration: 3 bags each time

### Example 3

### The Preparation of Tablets of the Pharmaceutical Composition According to the Present Invention

300 g Astragalus root, 200 g Pleurotus sapidus, 500 g Winnged Euonymus Twig and 50 g Mulberry Leaf were weighed respectively and decocted together for two hours with 11 L water every time and for three times. The obtained extractives were combined and filtrated. The gained filtrate was concentrated to relative density between 1.03 and 1.04 at 60□. The concentrated filtrate was stood overnight. Then the filtrate was filtrated again using a 200-mesh-filter. The obtained filtrate was centrifugalized and separated. The gained centrifugate was concentrated and then dried by spray to fine granules. 75 g dextrin and 100 g 85% ethanol were added to the granules to form soft material. The soft material was filtrated using a 200-mesh-filter and dry at 60-65□ by air. After adding 0.8% magnesium stearate, the material was pressed into tablets. Each tablet weighed 1 g.

Direction for administration: 5-15 tablets each time

### Example 4

### The Preparation of Capsules of the Pharmaceutical Composition According to the Present Invention

200 g Astragalus root, 350 g Pleurotus sajor, 350 g Winnged Euonymus Twig and 120 g Mulberry Leaf were weighed respectively. Then the Astragalus root, Winnged Euonymus Twig and Mulberry Leaf were decocted together for two hours with 10 L water every time and for three times. The obtained extractives were combined to be the mixed extractive “a”. Pleurotus sajor was singly extracted for 2 hours at 70□ with 1 L 75% ethanol each time and for 3 times. The obtained extractives were combined to be the extractive “b”. Then the mixed extractive “a” and the extractive “b” were combined and filtrated. The gained filtrate was concentrated to relative density between 1.04 and 1.60 at 60□. The concentrated filtrate was stood overnight. Then the filtrate was filtrated using a 200-mesh-filter. The obtained filtrate was centrifugalized and separated. Then the filtrate was filtrated again using a 200-mesh-filter. The obtained filtrate was centrifugalized and separated. The gained centrifugate was concentrated and then dried by spray to powder. The powers were filled to be No. 0000 capsule, Each capsule was filled with 5 g drug powders.

Direction for administration: 10-30 capsules each time

### Example 5

### The Preparation of Oral Liquid of the Pharmaceutical Composition According to the Present Invention

50 g Astragalus root, 1000 g Pleurotus ostreatus, 100 g Winnged Euonymus Twig and 30 g Mulberry Leaf were weighed respectively. They were extracted for 1.5 hours with 12 L 70% ethanol each time at 70□ and for 3 times. The obtained extractives were combined and filtrated. The gained filtrate was concentrated to relative density between 1.03 and 1.04 at 60□ and the ethanol was recovered. The concentrated filtrate was stood overnight. Then the filtrate was filtrated again using a 200-mesh-filter. The obtained filtrate was centrifugalized and separated. The gained centrifugate was filled into 10 ml vials of the oral solution and sterilized at 130□.

Direction for administration: 1-3 vials each time

### Test Examples

I. Acute Toxicity Test of the Pharmaceutical Composition According to the Present Invention

Granules of example 1 were dissolved in water to be a solution with the highest concentration of 3.57 g crude drug/ml), that is, the highest concentration of the drug which can pass freely through the syringe needle for gastric perfusion of mice.

40 mice (Grade 2 ICR) were selected, which were provided by Beijing Weitong Lihua Experimental Animal Technologic Limited Company. Half of the mice were male and the other half were female. Each of the mice weights 19 B 23 g. The number of certificate for them was No. 0004, J. D.X.Zi. (2000) and general number was 049. The mice were made to fast for 12 h before the test.

The mice were divided into two groups at random according to their weight. One was the control group and the other was the drug administered group. Each of the group had 20 mice. Half of the mice were male and the other half were female. The drug was administered to the mice of the drug administration group by oral administration (i.g.) and the Volume of drug solution given was 40 ml/kg. The same volume of water was given to the mice of the control group. The drug was again given to the mice of the drug administration group after six hours and given the drug twice in all. After the administration of drug solution or water, the general conditions of all mice, such as the change of activity, body weight and food intake, were observed

Observation: All the abnormal reactions and the data of all mice were recorded in detail and observed continuously for 7 days, and observed if there was dead mouse. Macroscopic anatomical examinations were carried out on the dead mice.

After drug administration, any adverse reaction in the mice was not found. Abnormal change in body weights of the mice was not found for 7d continuously and abnormal reaction in other items of general conditions was not found too. None of the mice died. The weight change of mice before and after drug administration was showed in Table 1.

TABLE 1
the weight change of mice before and after drug administration by ig ( [symbol] s, n = 20)

| | | | | Body weight in d 3 | | Body weight in d 7 | |
|---|---|---|---|---|---|---|---|
| | Dosage | Body weight before | | after drug | | after drug | |
| | (g crude | drug administration | | administration | | administration | |
| Groups | drug/kg) | female | male | female | male | female | male |
| Control | — | 18.7 ± 0.6 | 19.8 ± 1.4 | 23.2 ± 1.2 | 24.5 ± 1.6 | 24.5 ± 0.8 | 29.5 ± 1.6 |
| group | | | | | | | |
| Drug | 286 | 18.5 ± 0.8 | 19.7 ± 0.9 | 22.9 ± 0.8 | 25.0 ± 1.0 | 23.8 ± 1.0 | 30.0 ± 2.0 |
| administered | | | | | | | |
| group | | | | | | | |

Compared with the control group, the value of p is more than 0.05(P>0.05).

According to the test, the LD₅₀ of the drug could not be found, that is, the drug had not acute toxicity.

II. The Test of Treatment Aids by Using the Pharmaceutical Composition According to the Present Invention

Activity tests and clinical trials of said pharmaceutical composition oil the anti-HIV-1 activity in vitro were carried out. The results were as follows:

(I) Activity Tests on the Anti-HIV-1 Activity in Vitro

Reagents and Solutions

(1) Reagents

MTT (3,(4,5-dimethylthiazol-2-yl)-2,5-diphenyl tetrazolium bromide); SDS (Sodium Dodecyl Sulfate); DMF (N,N′-Dimethyl formanine).

(2) Solutions

RPMI-1640 complete culture medium containing 10% serum of new-born calf (hyclone); 2 mM L-Glutamine, 10 Mm hepes free acid, 50□M 2-mercaptoethanol, 100,000IU Penicillin, 100□g Streptomycin sulfate.

Drugs

The granules of example 1 were dissolved in said culture medium, in which the germs were removed by filtration. Then the drug was stored under 4□ with the concentration of 2.5 mg/ml.

Cells and Viruses

C8166 and HIV-1 B/H9 cells were given as a gift by the English Medical Research Council, AIDS Reagent project. HIV-1 B was prepared by routine method. TCID₅₀ of the viruses was determined by titration method. The virus stock solution was stored under B70□ after it was divided and packed. The cells and viruses were both frozen and revivified by routine method.

The Cytotoxic Effect on C8166 Cells Produced by the Granules of Example 1

100□l C8166 cell suspension with the concentration of 3×10⁵ cells/ml was mixed with the drug solution of example 1 under different concentrations. And at the same time, the same volume of blank control hole without the drug and the control hole with positive drug AZT were set. They were put into 5% CO₂ incubator under 37□ for three days. The survival rate of cells was determined by MTT method. OD values 595 nm/630 nm) were determined by ELx800ELISA Reader. TC₅₀, which is the concentration of drug causing the death of 50% the cells, was determined.

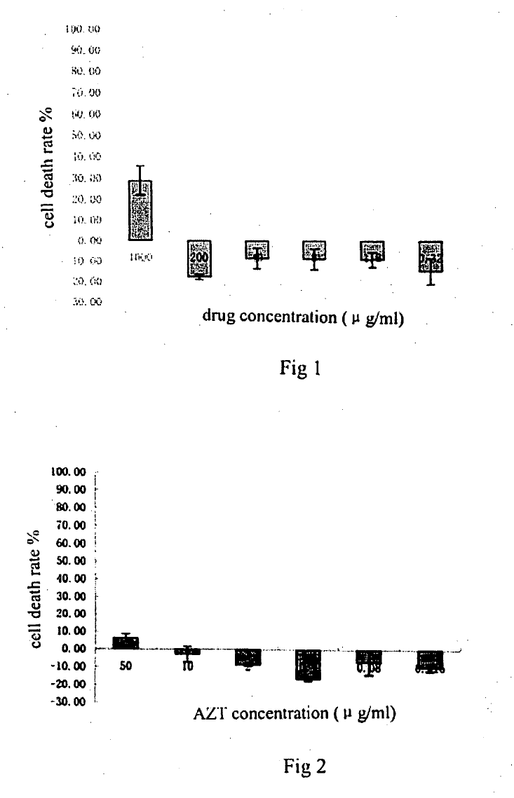

In Table 2 TC₅₀ of the granules of example 1 was listed. In FIG. 1, the toxic effects of the drugs with different concentrations on C8166 were showed. In FIG. 2, the toxic effects of AZT with different concentrations on C8166 were showed.

TABLE 2

TC₅₀ (μg/ml)

| Date of experiment | AZT | Granules of example 1 |
|---|---|---|
| Sep. 16, 2002 | >50 | >200 |
| Sep. 22, 2002 | >50 | 497.77 |
| Oct. 13, 2002 | >50 | >1000 |

The Inhibiting Effect of Granules of Example 1 on Syncytium Formation of C8166 Cells Induced by HIV-1

50□l C8166 cells with the concentration of 6×10⁵ cells/ml were mixed with 100□l drug solution of example 1 under different concentrations. 50□l diluted supernatant fluid of HIV-1 was added to the mixture and the value of M.O.I was 0.0089. At the same time, the control hole without the drug and the control hole with positive drug AZT were set. They were put into 5% CO₂ incubator under 37□ for three days. The amount of syncytium was counted under an inverted microscope (100×□) (in five visual fields). IC₅₀, that is, the concentration of the drugs inhibiting 50% syncytium formation, was determined. Selective index (SI) of the drug was calculated, the value of which is equal to that of TC₅₀/IC₅₀.

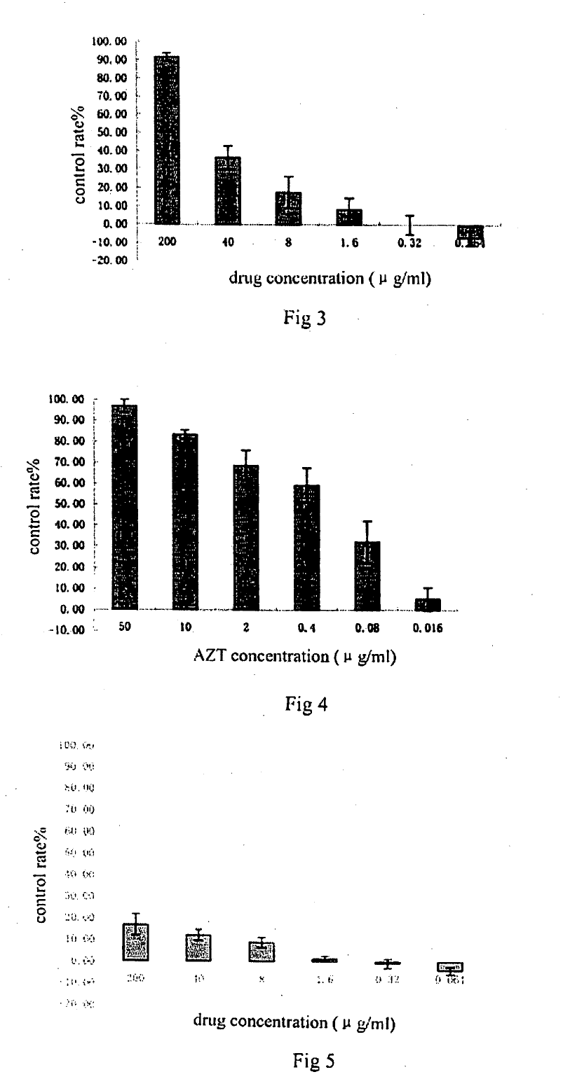

In table 3, IC₅₀ of the granules of example 1 inhibiting Syncytium formation was listed. In FIG. 3, the inhibiting effects of the granules of example 1 with different concentrations on Syncytium formation were showed. In FIG. 4, the inhibiting effects of AZT with different concentrations on Syncytium formation were showed.

TABLE 3

TC₅₀ (μg/ml)

| Date of experiment | AZT | Granules of example 1 |
|---|---|---|
| Sep. 16, 2002 | 0.23 | 59.36 |
| Sep. 22, 2002 | 0.18 | 49.41 |

Co-Cultivation Experiment

50□l C8166 cells under the concentration of 3×10⁵ cells/ml were mixed with 100□l drug solution of example 1 under different concentrations. 50□l H9 cells infected by HIV-1 were added into the mixture. At the same time, the control hole without the drug and the control hole with positive drug Dextran SulfateDS were set. They were put into 5% CO₂ incubator under 37□ for three days. The amount of syncytium was counted under an inverted microscope (100×□) (in five visual fields).

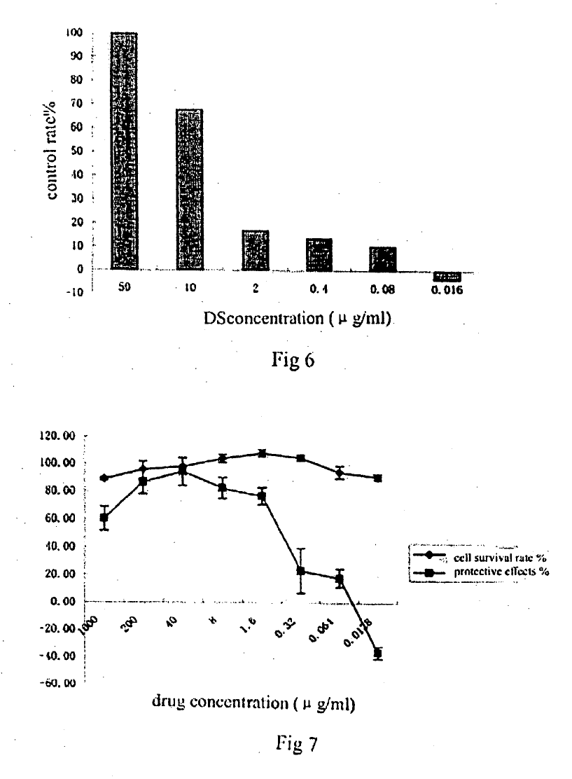

In table 4, IC₅₀ inhibited fusion by the granules of example 1 was listed. In FIG. 5, the inhibiting effects of the granules of example 1 under different concentrations on fusion were showed. In FIG. 6, the inhibiting effects of Dextran Sulfate(DS) under different concentrations on fusion were showed.

TABLE 4
IC₅₀ inhibited fusion by the granules of example 1

TC₅₀ (μg/ml)

| Date of experiment | DS | Granules of example 1 |
|---|---|---|
| Sep. 29, 2002 | 5.71 | >200 |

The Protective Experiment of the Granules of Example 1 on the Death of H9Cells Infected by HIV-1

50□l C8166 cells under the concentration of 6×10⁵ cells/ml were mixed with 100□l drug solution of example 1 under different concentrations. 50□l medium was added into half of the holes on the culture plate and 50□l diluted supernatant fluid of HIV-1, in which the value of M.O.I was 0.0089, was added into the other half of the holes on the culture plate. At the same time, the blank control holes without the drug (the negative control) were set and the control culture holes with positive drug AZT (the positive control) were set too. They were put into 5% CO₂ incubator under 37□ for three days. The survival rate of cells was determined by MTT method. OD values (595 nm/630 nm) were determined by ELx800ELISA Reader. The following formulae were used to calculate the survival rate of normal cells caused by the drug and the protective rate of the drug for HIV-1-infected cells.
Survival rate of normal cells(%)=OD value of the experimental hole/OD value of negative control hole□100%
Protective rate of infected cells(%)=(OD value of the experimental hole−OD value of positive control hole)/(OD value of negative control hole−OD value of positive control hole)□100%

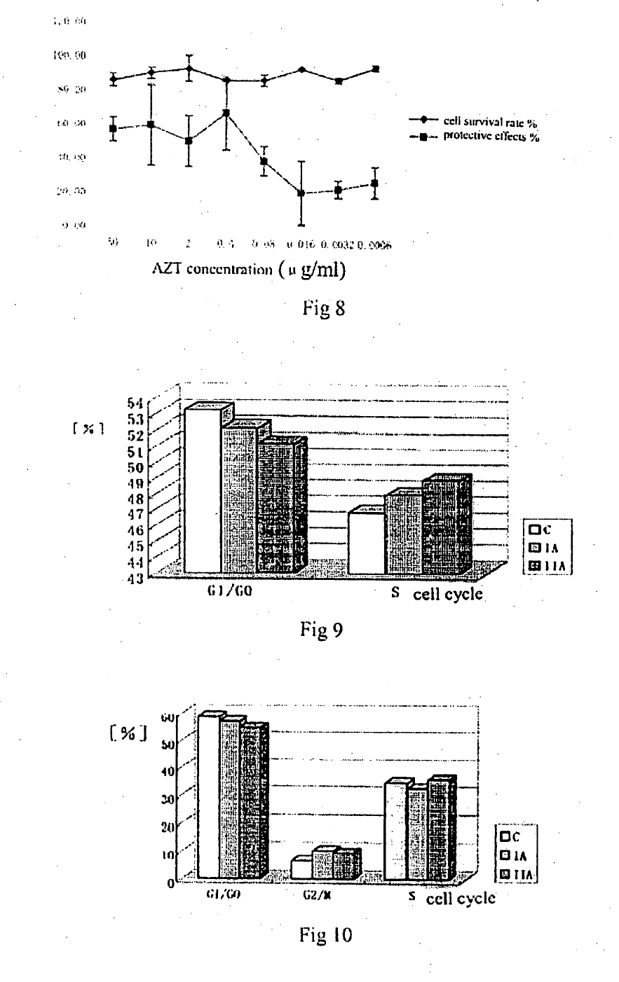

In table 5, the IC₅₀ of the toxic effects of the granules of example 1 on normal cells and the EC₅₀ of protective effects of the granules of example 1 on infected cells were listed. In FIG. 7, the protective effects of the granules of example 1 under different concentrations on infected cells were showed. In FIG. 8, the protective effects of AZT under different concentrations on infected cells were showed.

TABLE 5

| Drug (Oct. 13, 2002) | TC₅₀ (μg/ml) | IC₅₀ (μg/ml) |
|---|---|---|
| Granules of example 1 | >1000 | 0.71 |
| AZT | >50 | 0.15 |

According to the results of above experiments, the cytotoxic effect of the granules of example 1 on C8166 cells was relatively small. TC₅₀ of the drug was larger than 1000□g/ml. The IC₅₀ on the inhibition of syncytium formation of the host cells induced by HIV-1 was 54.64□g/ml (mean value) and selective index (SI) was greater than 18. TC₅₀ obtained by protective experiment was greater than 1000□g/ml and IC₅₀ obtained by protective experiment was 0.71□g/ml and selective index obtained by protective experiment was more than 1400. The granules of example 1 did not have blocking effect on the fusion between HIV-1-infected cells and normal cells and IC₅₀ obtained was more than 200□g/ml. It was obvious that the granules of example 1 had anti-HIV-1 activity to a certain extent in vitro.

(II) Clinical Trials

The granules of example 1 were used in the therapy of under-mentioned 10 patients for 10 months from Sep. 1, 2002 to Jun. 30, 2003. The results were as follows:

10 AIDS patients were selected at random who were diagnosed according to diagnostic criteria of AIDS by World Health Organization (WHO), that is, HIV positive, the CD4 cells count below 250 cells/mm³. Wherein, 6 patients were male and 4 patients were female at the age of 33-55 and average age of 42.4. All of them had AIDS for 1-4 years and 2.3 years on average. All the patients were infected by infusion.

Material and Methods

All the AIDS patients were observed in Clinic. A standard table about the conditions of each patient to be observed was filled before observing. The patients were visited by specific person once every week. The symptoms, adverse reactions and toxic effects in detail after taking the drugs were recorded.

The venous blood of patients was withdrawn for the HIV (COPY/ml) test and the CD4(mm³) test before and after the therapy. The blood samples were tested in the AIDS Clinical Trials Research Center of Beijing Tiantan Hospital.

Direction for administration: the patients took the granules of example 1 at 30 minutes before meal with warm water, took three times per day and 3 bags each time.

Full-time nurses provided service to the patients everyday and recorded adverse reactions and toxic effects after taking the granules.

Table 6-9 showed the symptom relief and the blood test results of the patients.

TABLE 6
average relief of the ten patients after therapy

| time | | Before | After therapy | | | | | | | | | |
|---|---|---|---|---|---|---|---|---|---|---|---|---|
| item | | therapy | 1 | 2 | 3 | 4 | 5 | 6 | 7 | 8 | 9 | 10 |
| weight | | 61.9 kg | | | | | | | | | | 68.9 kg |
| temperature | | 38.9□ | 37.6 | 37.2 | 36.9 | 36.2 | 36.4 | 36.1 | 36.2 | 36.2 | 36.2 | 36.2 |
| appetite | | poor | better | better | increase | increase | increase | − | − | − | − | − |
| weakness | | worst | worse | relief | recovery | recovery | recovery | − | − | − | − | − |
| Cough | | +++ | ++ | ++ | +/− | − | − | − | − | − | − | − |
| chest tightness | | +++ | ++ | ++ | +/− | − | − | − | − | − | − | − |
| Diarrhoea(time/day) | | 0-4 | 1-4 | 1-2 | 1 | 1 | normal | normal | normal | normal | normal | normal |
| Other | | Skin | + | + | + | + | − | − | − | − | − | − |
| | | rash | | | | | | | | | | |
| Drug | Nausea | − | − | − | − | − | − | − | − | − | − | − |
| Reaction | Skin | − | − | − | − | − | − | − | − | − | − | − |
| | rash | | | | | | | | | | | |
| | headache | − | − | − | − | − | − | − | − | − | − | − |
| | other | − | − | − | − | − | − | − | − | − | − | − |

TABLE 7
the change of HIV and CD4 content of the ten patients during therapy

| | Time(m) | Before | After therapy (/m) | | | | | |
|---|---|---|---|---|---|---|---|---|
| No. | Item | therapy | 1 | 2 | 3 | 4 | 5 | 6 |
| 1 | HIV copy/ml | | | | | | | |
| | CD4/mm³ | 140 | | | | | | 270 |
| 2 | HIV copy/ml | 17000 | 35000 | 11000 | 11000 | | | 4300 |
| | CD4/mm³ | 140 | 400 | 420 | 240 | | | 260 |
| 3 | HIV copy/ml | 1900000 | 1800000 | 1700000 | 1400000 | | | <LDL |
| | CD4/mm³ | 100 | 130 | 110 | 160 | | | 100 |
| 4 | HIV copy/ml | 180000 | 46000 | 130000 | 330000 | | | 350000 |
| | CD4/mm³ | 180 | 180 | 150 | 200 | | | 120 |
| 5 | HIV copy/ml | 35000 | 8900 | 7500 | 160000 | | | 43000 |
| | CD4/mm³ | 120 | 160 | 220 | 120 | | | 120 |
| 6 | HIV copy/ml | 710000 | 320000 | 420000 | 450000 | | | 1100000 |
| | CD4/mm³ | 100 | 120 | 100 | 130 | | | 80 |
| 7 | HIV copy/ml | 2600000 | 9300 | 1000000 | 1500000 | | | 2500000 |
| | CD4/mm³ | 80 | 200 | 180 | 180 | | | 180 |
| 8 | HIV copy/ml | 160000 | 110000 | 110000 | 260000 | | | |
| | CD4/mm³ | 80 | 140 | 90 | 120 | | | 80 |
| 9 | HIV copy/ml | 200000 | | 200000 | 390000 | | | 160000 |
| | CD4/mm³ | 220 | | 200 | 220 | | | 340 |
| 10 | HIV copy/ml | 46000 | 61000 | 74000 | 65000 | | | 220000 |
| | CD4/mm³ | 180 | 150 | 120 | 170 | | | 140 |

TABLE 8
routine test results of the ten patients after 10 months of therapy

| | No. item | | | | | | | | | |
|---|---|---|---|---|---|---|---|---|---|---|
| | 1 | 2 | 3 | 4 | 5 | 6 | 7 | 8 | 9 | 10 |
| Hbg/l | 115 | 105 | 115 | 120 | 115 | 120 | 105 | 110 | 130 | 107 |
| WBc10⁹/l | 9.2 | 8.6 | 9.8 | 10 | 8.8 | 9.6 | 9.0 | 8.5 | 7.2 | 8.0 |
| Pc %/N | 0.69 | 0.75 | 0.72 | 0.76 | 0.74 | 0.70 | 0.75 | 0.72 | 0.68 | 0.70 |
| L | 0.31 | 0.25 | 0.38 | 0.24 | 0.26 | 0.30 | 0.25 | 0.28 | 0.29 | 0.30 |

After taking the granules of example 1 for 10 months, Hb and WBc of patients were within the normal range, that is, no distinct evidence showed that the granules of example 1 make destroying effect on hemopoietic system of bone marrow and no distinct evidence showed that patients present inflammation.

TABLE 9
chest x-ray results of the ten patients after 10 months of therapy

| | No. item | | | | | | | | | |
|---|---|---|---|---|---|---|---|---|---|---|
| | 1 | 2 | 3 | 4 | 5 | 6 | 7 | 8 | 9 | 10 |
| Chest X-rays | (−) | − | + | − | − | − | − | − | − | − |
| Chest marking | (−) | − | + | + | + | + | + | + | + | + |
| Punctate and patchy | − | +/− | − | − | − | − | − | − | − | − |
| shadow/diffuse | | | | | | | | | | |
| patchy shadow | | | | | | | | | | |
| Heart | − | − | − | − | − | − | − | − | − | − |

Of the 10 patients, clinical symptoms before therapy included fever, cough, chest tightness, weakness, diarrhoea, skin rash, anorexia, nausea, weight loss and so on. After 4-6 months of therapy, body temperature of the patients was decreased from 38.9 (mean) to 36.2 (mean). 3 cases confined to the sickbed for long time got a normal life. All the patients could work. After 7 months of therapy, the chest x-rays results showed that 8 cases had punctate and patchy shadow and 1 case had diffuse patchy shadow that belonged to PCP—pneumocystis carinii pneumonia. After 10 months, the chest x-ray showed that the punctate and patchy shadows of the 8 cases were disappeared and the diffuse patchy shadow of the 1 case was absorbed evidently. Blood routine test after 10 months of therapy testified that total count and differentiation of white blood cell were within the normal range and the lung infections of the AIDS patients were controlled, which accorded with the fact of symptom relief of the cough and chest tightness. The haemoglobin was within the normal range. It testified that the granules of example 1 had no destroying effect on hematopoiesis in bone marrow. Clinical adverse reactions and toxic effects were not observed.

From the above-mentioned contents, it was obvious that the administration of the granules of example 1 could relieve obviously the symptoms of AIDS patients, build up health and improve the life quality, which may be associated with an increase in the count of T lymphocyte in peripheral blood, an increase in the count of helper T lymphocyte markedly, an decrease in the count of suppressor T lymphocyte, an improvement in forming antibody by T lymphocyte, an improvement in T lymphocyte, an improvement in immune function. Then the increase in the count of CD4 cells in clinic leaded to decrease Viral Load and had a good effect on the prevention and treatment of AIDS.

III. Clinical Trials of Treatment Diabetes by Using the Pharmaceutical Composition According to the Present Invention

According to diagnostic criteria of diabetes by WHO (1985), 104 diabetes patients were diagnosed to have type 2 diabetes. Wherein, 56 patients were male and 48 patients were female at average age of 49.19.4. All of them had diabetes for 10.472.53 years. They were divided into two groups at random:

Group A: treatment group administrated the granules of example 1

Group B: control group administrated Micronase (produced by Shandong Boshan Pharmaceutical Factory, Batch: 990917)

Testing Method

Therapy: for 3 months

Direction for administration:

Treatment group: granules of example 1 were orally taken at 30 minutes before meal in three times per day and 3 bags each time.

Control group: Micronase was orally taken at 30 minutes before meal in three times per day and 2.5 mg (tablet) each time.

TABLE 10
showed the effect of drugs in the
two groups on lowing blood sugar.

| | | Mean before | Mean after | |
|---|---|---|---|---|
| Time item | | therapy | therapy | P |
| fasting | treatment group | 9.41 ± 2.78 | 6.47 ± 1.74 | <0.05 |
| blood | (52 cases) | | | |
| sugar | Control group | 9.53 ± 1.46 | 6.53 ± 1.53 | <0.05 |
| | (52 cases) | | | |
| | P | >0.05 | >0.05 | |
| blood | treatment group | 14.64 ± 1.57 | 10.49 ± 1.34 | <0.05 |
| sugarat 2 | (52 cases) | | | |
| hours | Control group | 14.67 ± 1.49 | 12.14 ± 2.53 | <0.05 |
| after meal | (52 cases) | | | |
| | P | >0.05 | <0.05 | |

Thus, the granules of example 1 had a good effect on lowing blood sugar and had curative effect on diabetes.

IV. The Test of Inhibiting Tumor Growth in Vitro by Administrating the Pharmaceutical Composition According to the Present Invention

Materials:

1. IA: powder of Pleurotus ostreatus

IIA: granules of example 1

2. cell line:

Cervical cancer HeLa, Neuroblastoma cell line SK, lung cancer A549 and Neuroblastoma cell line SY5Y were provided by cell culture room in Capital University of Medical Sciences.

3. culture medium:

DMEM (high-sugar), Hyclone, NaHCO₃, Double-antibody, PBS, 0.25% Trypsin, MTT, PI, 95% ethanol at −20□, DMSO.

4. 25 cm² culture bottle, 96 well culture plate, 24 well plate, 10 ml Conical Centrifuge Tubes

5. measure index:

Change of state: phase contrast microscope, HE staining

Change of growth inhibition

Change of cell cycle

Clone forming rate

Test Plans

1. MTT method

Effective concentration range and reaction time of said two drugs for inhibiting growth of tumor cell line were determined by screen test and then the statistics analysis was conducted.

2. The effect of effective concentration on cell morphology was observed with phase contrast microscope and by staining test.

3. The effect of drugs on cell cycle was analyzed by Flow Cytometry.

4. The inhibiting effect of drugs on the stem cells, which were out of cell proliferation cycle, was analyzed by clone forming rate.

Test Methods

(I) MTT method

1. 100 mg drug IA and drug IIA were dissolved in 10 ml DMEM culture medium respectively to be solution at the concentration of 10 mg/ml. The solution was divided into 1 ml volumes, stored respectively and then diluted by times.

2. Cells in logarithmic growth phase were digested by 0.25% Trypsin to obtain single-cell suspension. After the cells were counted, the suspension was diluted to be the one at the concentration of 8□10⁴/ml.

3. The diluted suspension was inoculated into 96 well culture plate. After preincubation for 24 hours, the culture medium was discarded and then the test groups were divided. Different drug was respectively added to the different group.

4. grouping:

| A1 | 2 | 3 | 4 | 5 | 6 | A7 | 8 | 9 | 10 | 11 | 12 |
|---|---|---|---|---|---|---|---|---|---|---|---|
| B | | | | | | B | | | | | |
| C | | | | | | C | | | | | |
| D | | | | | | D | | | | | |
| E1 | | | | | | E7 | | | | | |
| F | | | | | | F | | | | | |
| G | | | | | | G | | | | | |
| H | | | | | | H | | | | | |

The grouping rule was as follows:

IA: 1—control; 2—5 ng/m; l3—50 ng/ml; 4—500 ng/ml; 5—5 ug/ml; 6—50 ug/ml

IIA: 7—control; 8—5 ng/ml; 9—50 ng/ml; 10—500 ng/ml; 11—5 ug/ml; 12—50 ug/ml

plate 1SY5Y A-D HeLa E-H

plate 2SK A-D A549 E-H

Each plate was reacted for 24 hours, 48 hours and 72 hours respectively

4. 24 ul MTT liquid with the concentration of 5 mg/l ml was added to each well in the culture plate at 37□. After incubation for 4 hours, 200 ul DMSO was added to each well in the culture plate, which was mixed evenly. Then the absorbance at 600 nm was measured by ELIASA.

5. The difference among the various concentrations was calculated by SPSS Statistics software to find optimal reaction concentration and time.

6. The above test was repeated once.

(II) The Effect of IA and IIA on the Cell Morphology, the Cell Cycle and the Clone Forming Rate

1. Cells in logarithmic growth phase were digested by 0.25% Trypsin to obtain single-cell suspension. After the cells were counted, the suspension was diluted to be the one with the concentration of 8□10⁴/ml.

2. The diluted suspension was inoculated into 24 well culture plate with cover glass and was inoculated into 50 ml culture bottle too. After preincubation for 24 hours, the culture medium was discarded and then the test groups were divided. Drugs IA and IIA with optimal concentrations was respectively added to each group, which was incubated for a certain time.

3. grouping:

| A1 | 2 | 3 | 4 | 5 | 6 |
|---|---|---|---|---|---|
| B1 | | | | | |
| C1 | | | | | |
| D1 | | | | | |

4. After incubation for some time, the cells in 12 well culture plate were taken out and then analyzed by common phase contrast photography and staining photography.

5. The cells in culture bottle were digested into single-cell suspension. After the cells were counted, the suspension was diluted to be the one with the concentration of 8□10⁴/ml and then added into 6 well plate respectively.

Al-3: SY5Y B1-3: HeLa C1-3: SK D1-3□A549

1—control group 2—drug IA, 3—drug IIA

After incubation for 1-2 weeks at 37□, the clone forming rate was counted by staining

The cell suspension of step 5 was fixed by 95% ethanol for 12 hours and then stained by PI. The change of cell cycle was analyzed by flow cytometry.

Results:

Growth Inhibition of Tumor Cell Line

Table 11 showed the inhibiting effect of IA on cell proliferation.

TABLE 11
the inhibiting effect of IA on cell proliferationOD = xSDn = 4

| | time | control | 50 ng/ml | 500 ng/ml | 5 □g/ml | 50 □g/ml | 500 □g/ml |
|---|---|---|---|---|---|---|---|
| HeLa | 24 h | 0.409 ± 0.01 | 0.216 ± 0.01* | 0.212 ± 0.01* | 0.209 ± 0.01* | 0.238 ± 0.006* | 0.396 ± 0.01 |
| | 48 h | 0.588 ± 0.01 | 0.273 ± 0.008* | 0.278 ± 0.007* | 0.283 ± 0.003* | 0.336 ± 0.01* | 0.624 ± 0.01 |
| | 72 h | 0.915 ± 0.01 | 0.430 ± 0.01* | 0.420 ± 0.01* | 0.425 ± 0.01* | 0.528 ± 0.01* | 0.924 ± 0.11 |
| SK | 24 h | 0.299 ± 0.01 | 0.301 ± 0.01 | 0.305 ± 0.01 | 0.259 ± 0.01* | 0.292 ± 0.006 | 0.367 ± 0.01 |
| | 48 h | 0.639 ± 0.01 | 0.582 ± 0.01* | 0.562 ± 0.007* | 0.575 ± 0.003* | 0.659 ± 0.01 | 0.709 ± 0.01 |
| | 72 h | 0.687 ± 0.01 | 0.483 ± 0.01* | 0.484 ± 0.01* | 0.584 ± 0.01* | 0.714 ± 0.01 | 0.788 ± 0.01 |
| SY5Y | 24 h | 0.498 ± 0.02 | 0.354 ± 0.02* | 0.351 ± 0.01* | 0.341 ± 0.02* | 0.368 ± 0.02* | 0.507 ± 0.03 |
| | 48 h | 0.914 ± 0.07 | 0.463 ± 0.04* | 0.443 ± 0.04* | 0.443 ± 0.04* | 0.478 ± 0.003* | 0.781 ± 0.03* |
| | 72 h | 1.076 ± 0.07 | 0.829 ± 0.05* | 0.767 ± 0.07* | 0.767 ± 0.08* | 0.798 ± 0.10* | 1.081 ± 0.04 |
| A549 | 24 h | 0.339 ± 0.02 | 0.352 ± 0.007* | 0.349 ± 0.009* | 0.339 ± 0.003* | 0.331 ± 0.01* | 0.365 ± 0.02* |
| | 48 h | 0.439 ± 0.03 | 0.431 ± 0.009 | 0.388 ± 0.04* | 0.372 ± 0.02* | 0.366 ± 0.02* | 0.406 ± 0.02 |
| | 72 h | 0.680 ± 0.06 | 0.556 ± 0.03* | 0.536 ± 0.04* | 0.561 ± 0.05* | 0.573 ± 0.06* | 0.691 ± 0.02 |

Thus,

(1) IA had an inhibiting effect on Hela cell proliferation. The drugs under the concentrations of 50 ng/ml, 500 ng/ml, 5□g/ml and 50□g/ml had obvious inhibiting effect on cell proliferation after acting continuously oni cells for 24, 48 or 72 hours, but the effect is not positively related to the drug doses. When the drug concentration is 500 ng/ml, the drug has the effect of promoting cell proliferation. The optimal time acting with Hela cells is 24-hours and the optimal concentration is 5□g/ml.

(2) IA had weak inhibiting effect on SK cell proliferation. There is significant difference in growth inhibition effect between the various drug groups and the control group when the drug concentration is 5 ug/ml and reaction duration is 24 h, 48 h or 72 h. When the drug concentration is 500 ug/ml, the drug has the effect of promoting cell proliferation. For SK cell proliferation inhibition, the optimal duration is 24 h and the optimal drug concentration is 5 ug/ml.

(3) IA had an inhibiting effect on SY5Y cell proliferation. 4 groups of the drug concentrations (50 ng/ml, 500 ng/ml, 5 ug/ml and 50 ug/ml) and consecutive reaction duration of 24 h, 48 h and 72 h on SY5Y cells have shown obvious inhibition effect on cell proliferation, but the effect is not positively related to the drug dose. When the drug concentration is 500 ug/ml, the drug has the effect of promoting cell proliferation. For SY5Y cell proliferation inhibition, the optimal duration is 24 h, and the optimal drug concentration is 5 ug/ml.

(4) IA had an inhibiting effect on A549 cell proliferation. 4 groups of the drug concentrations (50 ng/ml, 500 ng/ml, 5 ug/ml and 50 ug/ml) and consecutive reaction duration of 24 h, 48 h and 72 h on A549 cells have obvious inhibition effect on cell proliferation, but the effect is not positively related to the drug dose. When the drug concentration is 500 ug/ml, the drug has the effect of promoting cell proliferation. For SY5Y cell proliferation inhibition, the optimal duration is 24 h and the optimal drug concentration is 5 ug/ml.

Table 12 showed the inhibiting effects of IIA on cell proliferation

TABLE 12
the inhibiting effects of IIA on cell proliferationOD = xSDn = 4

| | time | control | 50 ng/ml | 500 ng/ml | 5 □g/ml | 50 □g/ml | 500 □g/ml |
|---|---|---|---|---|---|---|---|
| HeLa | 24 h | 0.380 ± 0.01 | 0.378 ± 0.01 | 0.229 ± 0.01* | 0.235 ± 0.01* | 0.237 ± 0.006* | 0.354 ± 0.01 |
| | 48 h | 0.588 ± 0.01 | 0.586 ± 0.008 | 0.285 ± 0.007* | 0.279 ± 0.003* | 0.293 ± 0.01* | 0.371 ± 0.01* |
| | 72 h | 0.915 ± 0.01 | 0.857 ± 0.01 | 0.424 ± 0.01* | 0.391 ± 0.01* | 0.410 ± 0.01* | 0.349 ± 0.11* |
| SK | 24 h | 0.299 ± 0.01 | 0.233 ± 0.01* | 0.253 ± 0.01* | 0.273 ± 0.01* | 0.304 ± 0.006* | 0.634 ± 0.01 |
| | 48 h | 0.639 ± 0.01 | 0.527 ± 0.02* | 0.551 ± 0.04* | 0.554 ± 0.003* | 0.580 ± 0.01 | 0.566 ± 0.01 |
| | 72 h | 0.687 ± 0.01 | 0.698 ± 0.01 | 0.677 ± 0.01 | 0.775 ± 0.01 | 0.671 ± 0.01 | 0.832 ± 0.11 |
| SY5Y | 24 h | 0.498 ± 0.02 | 0.453 ± 0.02 | 0.354 ± 0.01* | 0.347 ± 0.02* | 0.363 ± 0.02* | 0.465 ± 0.03 |
| | 48 h | 0.914 ± 0.07 | 0.804 ± 0.07* | 0.463 ± 0.04* | 0.455 ± 0.03* | 0.469 ± 0.02* | 0.579 ± 0.04* |
| | 72 h | 1.076 ± 0.07 | 0.780 ± 0.07 | 0.748 ± 0.09 | 0.715 ± 0.09 | 0.784 ± 0.02* | 1.126 ± 0.42 |
| A549 | 24 h | 0.439 ± 0.02 | 0.370 ± 0.006* | 0.373 ± 0.02* | 0.382 ± 0.01 | 0.398 ± 0.01 | 0.693 ± 0.06 |
| | 48 h | 0.519 ± 0.01 | 0.451 ± 0.008* | 0.443 ± 0.007* | 0.458 ± 0.003* | 0.463 ± 0.01* | 0.509 ± 0.01 |
| | 72 h | 0.680 ± 0.05 | 0.579 ± 0.01* | 0.591 ± 0.01* | 0.616 ± 0.01 | 0.690 ± 0.01 | 0.86 ± 0.04 |

(1) IIA has inhibition effect on HeLa cell proliferation, 3 groups of the drug concentrations (500 ng/ml, 5 ug/ml and 50 ug/ml) and consecutive reaction duration of 24 h, 48 h and 72 h on HeLa cells have obvious inhibition effect on cell proliferation. When the drug concentration is 500 ug/ml and reacted continuously for 48 h and 72 h, the drug has a certain inhibition effect. For HeLa cell proliferation inhibition, the optimal duration is 72 h and the optimal drug concentration is 500 ug/ml.

(2) IIA has inhibition effect on SK cell proliferation, 4 groups of the drug concentrations (50 ng/ml, 500 ng/ml, 5 ug/ml and 50 ug/ml) and consecutive reaction duration of 24 h and 48 h on SK cells have obvious inhibition effect on cell proliferation. When the drug concentration is 500 ug/ml, the drug has no inhibition effect on cell proliferation; when the drug within the range of test concentrations reacted consecutively with SK for 72 h, it has no proliferation inhibition effect on SK cells. For SK cell proliferation inhibition, the optimal duration is 24 h and the optimal drug concentration is 500 ng/ml.

(3) IIA has inhibition effect on SY5Y cell proliferation, 3 groups of the drug concentrations (500 ng/ml, 5 ug/ml and 50 ug/ml) and consecutive reaction duration of 24 h and 48 h on SY5Y cells have obvious inhibition effect on cell proliferation. When the drug concentration is 500 ug/ml, the drug has no inhibition effect on cell proliferation; when the drug within the range of test concentrations reacted consecutively with SY5Y for 72 h, it has no proliferation inhibition effect on SY5Y cells. For SY5Y cell proliferation inhibition, the optimal duration is 24 h and the optimal drug concentration is 5 ug/ml.

(4) IIA (50 ng/ml and 500 ng/ml) reacted consecutively with A549 for 24 h reaction has obvious inhibition effect on A549 cell proliferation. 5 groups of the drug concentrations (50 ng/ml, 500 ng/ml, 5 ug/ml, 50 ug/ml and 500 ug/ml) and consecutive reaction duration of 48 h on A549 cells have obvious inhibition effect on A549 cell proliferation, but if they reacted consecutively with A549 cells for 72 h, only low concentrations (50 ng/ml and 500 ng/ml) have proliferation inhibition effect on such cells. For A549 cell proliferation inhibition, the optimal duration is 48 h and the optimal drug concentration is 500 ng/ml.

The Effect on Cell Cycle of Tumor Cell Line

The change of the cell cycle was analyzed by Flow Cytometry after IA and IIA with the concentration shown in table 13 reacted on the four cells for 24 hours.

TABLE 13
the reacting concentration of IA and IIA on the four cells

| | | |
|---|---|---|
| Drug Type | | |
| Cell Type | IA | IIA |

| | | | | |
|---|---|---|---|---|
| HeLa | 5 | ug/ml | 5 | ug/ml |
| SK | 5 | ug/ml | 500 | ng/ml |
| SY5Y | 5 | ug/ml | 5 | ug/ml |
| A549 | 50 | ug/ml | 500 | ng/ml |

The Results

The effects of IA and IIA on the cell cycle of HeLa cells: IA and IIA improved the cells in G0/G1 phase into S phase. The ratio of G0/G1 phase was decreased and the cells were blocked in S phase. The apoptosis did not happen under said concentrations. FIG. 9 showed the result, wherein C was control.

The effects of IA and IIA on the cell cycle of SK cells: IA and IIA improved the cells in G0/G1 phase into G2/M phase. The ratio of G2/M phase was increased and the change of S phase was not evident. The apoptosis did not happen under said concentrations. FIG. 10 showed the result, wherein C was control.

The effects of IA and IIA on cell cycle of SY5Y cells: IA and IIA improved the cells in G0/G1 phase to G2/M phase. The ratio of G2/M phase was increased and the change of S phase was not evident. The apoptosis did not happen under said concentrations. FIG. 11 showed the result, wherein C was control.

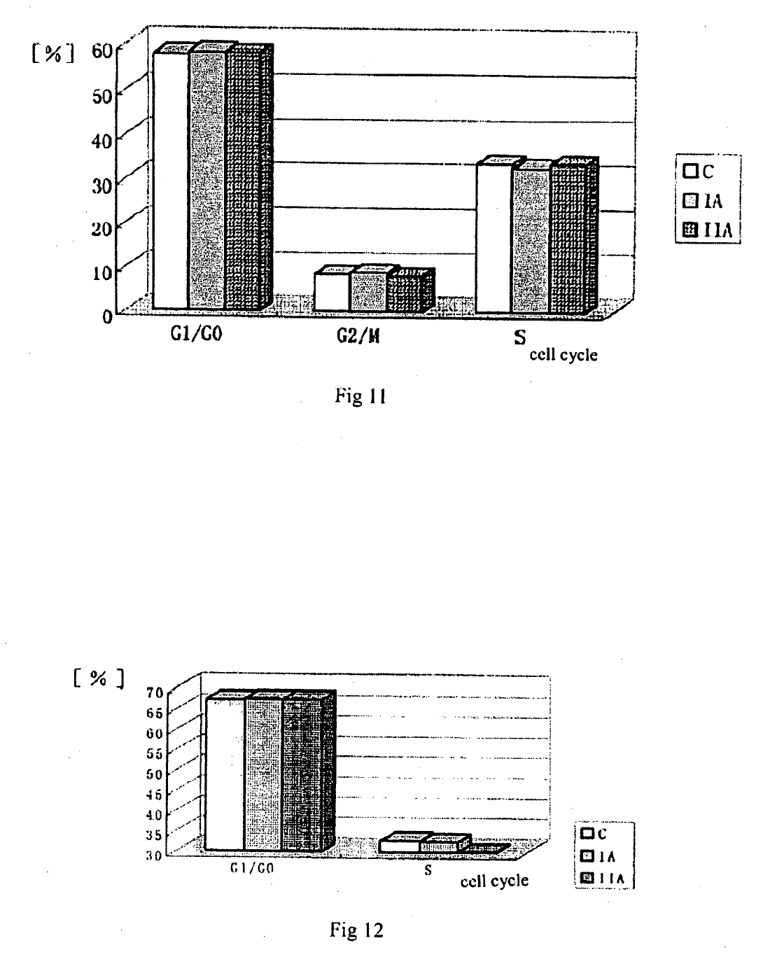

The effects of IA and IIA on cell cycle of A549 cells: The effect of IA and IIA on A549 cell cycle was not evident. A made A549 blocking in G0/G1 phase and the ratio of S phase was decreased. The apoptosis did not happen under said concentrations. FIG. 12 showed the results.

The Effects on Clone Forming Rate of Tumor Cell Line

IA and IIA under the concentrations shown in table 14 reacted with four kinds of tumor cells for 24 h and the effect of the drugs on clone forming rate was analyzed.

TABLE 14
the reacting concentration of IA and
IIA on the four kinds of tumor cells

| | | |
|---|---|---|
| Drug Type | | |
| Cell Type | IA | IIA |

| | | | | |
|---|---|---|---|---|
| HeLa | 5 | ug/ml | 5 | ug/ml |
| SK | 5 | ug/ml | 500 | ng/ml |
| SY5Y | 5 | ug/ml | 5 | ug/ml |
| A549 | 50 | ug/ml | 500 | ng/ml |

The effect of IA and IIA under the above concentrations on clone forming rate of four kinds of tumor cells was shown in table 15.

TABLE 15
clone forming rate of tumor cells under
the concentrations shown in table 14

| Drug | | | |
|---|---|---|---|
| Cell type | Control group | Group IA | Group IIA |
| HeLa | 29% | 14% | 27% |
| A549 | 19% | 14% | 9% |
| SK | 27% | 21% | 22% |
| SY5Y | 30% | 20% | 18% |

Thus, IA and IIA had some inhibiting effects on clone forming rate of the four kinds of tumor cells

From the test, it was obtained that IA and IIA had good inhibiting effects on proliferation of HeLa, SK, A549 and SY5Y cells. IA and IIA had some inhibiting effects on clone forming rate of said four kinds of tumor cells. The effects on HeLa and A549 cells in S phase were evident, which made the ratio of cells in S phase was decreased. But the effect on SK and SY5Y cells in S phase was not evident. So IA and IIA had a certain inhibiting effects on tumor cell growth.

While the invention has been described in terms of what are presently considered to be the most practical and preferred embodiments, it is to be understood that the invention need not to be limited to the disclosed embodiment. On the contrary, it is intended to cover various modifications and similar arrangements included within the spirit and scope of the appended claims which are to be accorded with the broadest interpretation so as to encompass all such modifications and similar structures.

1-13. (canceled)

14. A method for regulating immunity, wherein said method comprising:
administering to a patient in need of such regulation a pharmaceutically effective amount of a pharmaceutical composition comprising 5-60 parts by weight of Astragalus root, 10-100 parts by weight of Pleurotus fungi, 10-100 parts by weight of winnged euonymus twig, and 3-40 parts by weight of mulberry leaf.

15. A method for treating diseases caused by immune dysfunction, said method comprising:
administering to a patient in need of such treatment, a pharmaceutically effective amount of a pharmaceutical composition comprising 5-60 parts by weight of Astragalus root, 10-100 parts by weight of Pleurotus fungi, 10-100 parts by weight of winnged euonymus twig, and 3-40 parts by weight of mulberry leaf.

16. A method for treating AIDS, said method comprising:
administering to a patient in need of such treatment a pharmaceutically effective amount of the pharmaceutical composition comprising 5-60 parts by weight of Astragalus root, 10-100 parts by weight of Pleurotus fungi, 10-100 parts by weight of winnged euonymus twig, and 3-40 parts by weight of mulberry leaf.

17. A method for treating diabetes, said method comprising:
administering to a patient in need of such treatment a pharmaceutically effective amount of a pharmaceutical composition comprising 5-60 parts by weight of Astragalus root, 10-100 parts by weight of Pleurotus fungi, 10-100 parts by weight of winnged euonymus twin, and 3-40 parts by weight of mulberry leaf.

18. A method for treating cancer, said method comprising:
administering to a patient in need of such treatment a pharmaceutically effective amount of a pharmaceutical composition comprising 5-60 parts by weight of Astragalus root, 10-100 parts by weight of Pleurotus fungi, 10-100 parts by weight of winnged euonymus twig, and 3-40 parts by weight of mulberry leaf.
